(19) United States
(12) Patent Application Publication
(10) Pub. No.: US 2010/0174096 A1
(43) Pub. Date: Jul. 8, 2010

(54) Methods for Production of Optically Active Fluoropyrrolidine Derivatives

(75) Inventors: Teruo Umemoto, Westminster, CO (US); Rajendra P. Singh, Broomfield, CO (US)

Correspondence Address: SWANSON & BRATSCHUN, L.L.C., 8210 SOUTHPARK TERRACE, LITTLETON, CO, 80120, US

(73) Assignee: IM&T RESEARCH, INC., Denver, CO (US)

(21) Appl. No.: 12/647,973

(22) Filed: Dec. 28, 2009

Related U.S. Application Data
(60) Provisional application No. 61/142,567, filed on Jan. 5, 2009.

(51) Int. Cl. C07D 207/16

(52) U.S. Cl. 548/533; 548/531

(57) ABSTRACT

Useful industrial methods for producing optically active fluoropyrrolidine derivatives as useful fluorinated intermediates are disclosed.

### TECHNICAL FIELD

The invention relates to industrially practical methods for producing optically active fluoropyrrolidine derivatives.

### BACKGROUND OF THE INVENTION

Optically active fluoropyrrolidine derivatives are particularly useful fluorinated intermediate compounds for production of therapeutics such as dipeptidyl peptidase (DPP) IV inhibitors which are useful in the treatment of diabetes [see, for example, Bioorganic & Medicinal Chemistry, 2004, Vol. 12, pp. 6053-6061; Bioorganic & Medicinal Chemistry, 2008, Vol. 16, pp. 4093-4106; Bioorganic & Medicinal Chemistry Letters, 2007, Vol. 17, pp. 4167-4172; WO 03/002533 A2 (Smithkline Beecham); U.S. Pat. No. 7,348,346 B2 (Abbott Laboratories); and WO 2008/001195 A2 (GlenMark Pharm.)], peptide deformylase (PDF) inhibitor (a novel class of antimicrobial agents) [Organic Process Research & Development, 2008, Vol. 12, pp. 183-191] and other like compounds. However, as described in more detail below, there are a number of drawbacks in the conventional preparation of an optically active fluoropyrrolidine derivative.

In this regard, an illustrative production method includes: N-protected (2S,4S)-4-fluoroprolinonitrile [N-protected (2S,4S)-4-fluoropyrrolidine-2-carbonitrile] prepared by fluorination of N-protected (2S,4R)-4-hydroxyprolinonitrile with a substituted aminosulfur trifluoride such as diethylaminosulfur trifluoride [see, for example, WO 03/002533 A2 (Smithkline Beecham) and WO 2008/001195 A2 (GlenMark Pharm.)] (conventional Method 1 herein).

Further, N-protected (2S,4S)-4-fluoroproline methyl esters [N-protected methyl (2S,4S)-4-fluoropyrrolidine-2-carboxylate] were prepared by the following methods: fluorination of N-protected (2S,4R)-4-hydroxyproline methyl ester with a substituted aminosulfur trifluoride such as diethylaminosulfur trifluoride [see, for example, Tetrahedron Letters, Vol. 39 (1998), pp. 1169-1172] and morpholinoaminosulfur trifluoride [see, Tetrahedron, Vol. 58 (2002), pp. 8453-8459] (conventional Method 2 herein); fluorination of N-protected (2S,4R)-4-hydroxyproline methyl ester with CFClHCF₂N(C₂H₅)₂ (Yarovenko reagent) or CF₃CFHCF₂N(C₂H₅)₂ (Ishikawa reagent) at specified reaction temperature with or without addition of an alcohol [WO 2006/103986 A1 (Tosoh F-Tech, Inc.)] (conventional Method 3 herein); fluorination of N-protected (2S,4R)-4-hydroxyproline ester with CF₃CFHCF₂N(C₂H₅)₂ (Ishikawa reagent) in the presence of a HF-trapping agent [U.S. Pat. No. 7,279,584 B2 (Taisho Pharm.)] (conventional Method 4 herein); reaction of (2S,4R)-N-(tert-butoxycarbonyl)-4-hydroxyproline methyl ester with perfluoroalkanesulfonyl fluoride in the presence of a base [Jpn Kokai Tokkyo Koho JP 2005-126386 (Kyorin Pharm.)] (conventional Method 5 herein); fluorination of (2S,4R)-N-(tert-butoxycarbonyl)-4-hydroxyproline methyl ester with an α,α-difluoroalkylamine, such as 2,2-difluoro-1,3-dimethylimidazolidine, in the presence of a basic organic compound or in an organic solvent having a Lewis basic nature [Jpn Kokai Tokkyo Koho JP 2008-174509 (Mitsui Chemicals Inc.)] (conventional Method 6 herein); and substitution reaction of (2S,4R)-N-(tert-butoxycarbonyl)-4-(trifluoromethanesulfonyloxy)proline methyl ester, which was derived from (2S,4R)-N-(tert-butoxycarbonyl)-4-hydroxyproline methyl ester with trifluoromethanesulfonic anhydride, with tetrabutylammonium fluoride [see, for example, Journal of Fluorine Chemistry, Vol. 129 (2008), pp. 781-784] (conventional Method 7 herein).

Other optically active N-protected (2S,4R)-4-fluoroproline esters were prepared by fluorination of N-protected (2S,4S)-4-hydroxyproline esters with a substituted aminosulfur trifluoride such as diethylaminosulfur trifluoride and morpholinoaminosulfur trifluoride [see, for example, Tetrahedron Letters, Vol. 39 (1998), pp. 1169-1172; Tetrahedron, Vol. 58 (2002), pp. 8453-8459] (conventional Method 8 herein).

Optically active N-protected (3S)- or (3R)-3-fluoropyrrolidines were prepared by the following: fluorination of (3R)-N-benzyloxycarbonyl-3-hydroxylpyrrolidine with a substituted aminosulfur trifluoride such as diethylaminosulfur trifluoride [see, for example, U.S. Pat. No. 7,348,346 B2 (Abbott Laboratories)] (conventional Method 9 herein); and substitution reaction of (3R)- or (3S)-N-benzyloxycarbonyl-3-(toluenesulfonyloxy)pyrrolidine, which was derived from (3R)- or (3S)-N-benzyloxycarbonyl-3-hydroxypyrrolidne with toluenesulfonyl chloride, with spray-dried potassium fluoride (Synlett, 1995, pp. 55-57) (conventional Method 10 herein). In addition, it was reported that N-benzyl-3-hydroxypyrrolidine was fluorinated with 2,2-difluoro-1,3-dimethylimidazolidine to give N-benzyl-3-fluoropyrrolidine [Jpn Kokai Tokkyo Koho JP 2004-269365 (Mitsui Chemicals Inc.)] (conventional Method 11).

Conventional methods (1), (2), (8) and (9) require substituted aminosulfur trifluorides, which are very reactive and thermally unstable compounds of potentially explosive nature [see, for example, “Chemical Safety: Laboratory explosions”, Chem. & Eng. News, Vol. 57, No. 19, pp. 4-5 (1979); J. Fluorine Chem., Vol. 42, pp. 137-143 (1989)]. In addition, these aminosulfur trifluoride fluorinating agents strongly fume in the air. Therefore, the use of these fluorinating agents in an industrially large scale may result in severe handling (costs, time, etc.,) and safety problems and render these conventional Methods unsuitable.

Conventional Method (3) requires Yarovenko reagent, which cannot be stored for long periods of time due to its unstable nature. The reagent is prepared from CClF═CF₂ which is a toxic gas (bp −28.4° C.) and may have a potential risk of polymerization. In addition, Yorovenko reagent has a significant defect in that the obtained fluorinated products are contaminated with chlorinated products [see, WO 2006/103986 A1 (Tosoh F-Tech, Inc.)]. Removal of the chlorinated products from the fluorinated products is very difficult, i.e., costly, inefficient, time consuming, etc. Therefore, conventional Method (3) involves problems in handling, safety, and purity of the fluorinated products, and is therefore unsatisfactory for use in industrial application.

Conventional Methods (3) and (4) require Ishikawa reagent which is low in fluorination performance, resulting in relative high cost, because the number of fluorine atom(s) (in the molecule) used for the fluorination is ⅕ of the total number of the fluorine atoms contained in the fluorinating agent [CF₃CFHCF₂N(C₂H₅)₂].

Method (5) is furthermore low in cost performance because the number of fluorine atom used for the fluorination is ⅙˜ 1/24 of the total number of the fluorine atom(s) contained in the fluorinating agent [CF₃(CF₂)ₙSO₂F; n=1-10]. In addition, Method (5) has a significant defect in that it includes use of perfluoroalkane compounds of a long carbon chain. These long chain carbon groups are problematic and should be eliminated due to persistent pollutants that bioaccumulate (see Chemical and Engineering News, Jan. 30, 2006, p. 8).

Methods (6) and (11) utilize 2,2-difluoro-1,3-dimethylimidazolidine, which may have a racemization problem during the fluorination reaction process because the reaction is conducted at elevated temperature in the presence of a basic organic compound (due to relatively low reactivity of the α,α-difluoroalkylamine). When the product is contaminated with a racemized product, it is hard to get an optically pure product. Thus, it may require additional processes for the purification, resulting in high cost.

Methods (7) and (10) require two reaction steps and an expensive reagent such as trifluoromethanesulfonic anhydride for Method (7), again making the Methods high cost and unsuitable for industrial application.

As such, there are a number of drawbacks in practicing these conventional methods for preparation of optically active fluorinated intermediate compounds. As a result, problems with the production methods for the fluorinated intermediate compounds have made it difficult to prepare useful, highly pure therapeutics in a cost effective, timely, industrially applicable and safe fashion. Therefore, there is a need in the art for the development of a methodology which makes it possible to prepare the fluorinated intermediates safely, easily, cost effectively, and in industrially useful amounts.

The present invention is directed toward overcoming one or more of the problems discussed above.

### SUMMARY OF THE INVENTION

The present invention provides useful methods for preparing optically active fluoropyrrolidine derivatives having a formula (I):

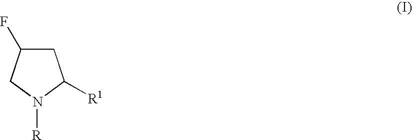

by reacting an optically active hydroxypyrrolidine derivative having a formula (II) with an arylsulfur trifluoride having a formula (III):

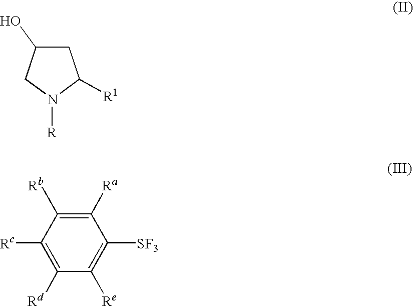

In formulas (I), (II), and (III), R is a protective group for an amino group; R¹ is a hydrogen atom, a cyano group, or a COOR² group, in which R² is a protective group for a carboxyl group; R^{a}, R^{b}, R^{c}, R^{d}, and R^{e} each is independently a hydrogen atom, a halogen atom, a substituted or unsubstituted alkyl group having one to ten carbon atoms, a nitro group, or a cyano group.

These and various other features as well as advantages which characterize embodiments of the invention will be apparent from a reading of the following detailed description and a review of the appended claims.

### DETAILED DESCRIPTION OF THE INVENTION

The invention provides methods for producing optically active fluoropyrrolidine derivatives, these compounds are useful intermediates for the production of various kinds of therapeutics, such as inhibitors and other like bioactive compounds. Particularly beneficial in this regard is the unexpected cost effective, industrial applicability of the methods herein.

Embodiments of the present invention provide methods for producing useful, optically active fluoropyrrolidine derivatives, as represented by formula (I):

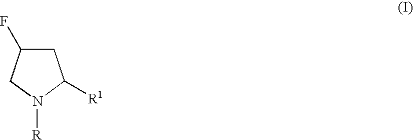

by reacting an optically active 4-hydroxypyrrolidine derivative having a formula (II) with an arylsulfur trifluoride having a formula (III):

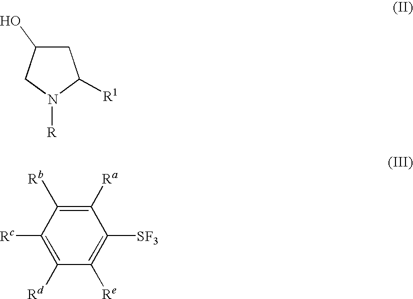

In formulas (I), (II), and (III), R is a protective group for an amimo group; R¹ is a hydrogen atom, a cyano group, or a COOR² group, in which R² is a protective group for a carboxyl group; R^{a}, R^{b}, R^{c}, R^{d}, and R^{e} each is independently a hydrogen atom, a halogen atom, a substituted or unsubstituted alkyl group having one to ten carbon atoms, a nitro group, or a cyano group. The halogen atom herein is a fluorine atom, chlorine atom, bromine atom, or iodine atom.

In preferred embodiments of formula (III), the substituted or unsubstituted alkyl group has one to four carbon atoms.

The term “alkyl” as used herein is linear or branched.

The term “protecting group” refers to a substituent that is employed to block or protect a particular functionality. Other functional groups on the compound may remain reactive. For example, a “protecting group of an amino group” is a substituent attached to an amino group that blocks or protects the functionality of the amino group in the compound. Preferable protecting groups of an amino group include, but are not limited to, acetyl, chloroacetyl, bromoacetyl, trifluoroacetyl, methoxycarbonyl, ethoxycarbonyl, tert-butoxycarbonyl (Boc), benzyloxycarbonyl (Cbz), 9-fluorenylmethoxycarbonyl (Fmoc), triphenylmethyl, benzyl, and substituted benzyl. For examples of protecting groups of an amino group and their use, see “Protective Groups in Organic Synthesis, 3^{rd} edition” by T. W. Greene and P. G. M. Wuts: pp. 494-653: John Wiley & Sons, Inc., New York 1999, incorporated by reference herein for all purposes.

Similarly, a “protecting group of a carboxyl group” is a substituent attached to a carboxyl group that blocks or protects the functionality of the carboxyl group in the compound. Preferable protecting groups of a carboxyl group include, but are not limited to, methyl, ethyl, tert-butyl, benzyl, 2-chloroethyl, 2-bromoethyl, 2-iodoethyl, 2,2,2-trichloroethyl, 2,2,2-trifluoroethyl, 9-fluorenylmethyl, phenyl, triphenylmethyl, phenacyl, and so on. For examples of protecting groups of a carboxyl group and their use, see “Protective Groups in Organic Synthesis, 3^{rd} edition” by T. W. Greene and P. G. M. Wuts: pp. 369-453: John Wiley & Sons, Inc., New York 1999, incorporated by reference herein for all purposes.

Optical activity and ability to measure optical activity are terms known in the art and their use herein is consistent with this use. The R,S system (Cahn-Ingold-Prelog or CIP system) is used to provide configuration of compounds herein and is also known in the art.

Preferable examples of compounds of formula (I) obtained by the present invention include: (2S,4S)-N-(9-fluorenylmethoxycarbonyl)-4-fluoroprolinonitrile, (2R,4R)-N-(9-fluorenylmethoxycarbonyl)-4-fluoroprolinonitrile, (2S,4R)-N-(9-fluorenylmethoxycarbonyl)-4-fluoroprolinonitrile, (2R,4S)-N-(9-fluorenylmethoxycarbonyl)-4-fluoroprolinonitrile, (2S,4S)-N-(benzyloxycarbonyl)-4-fluoroprolinonitrile, (2R,4R)-N-(benzyloxycarbonyl)-4-fluoroprolinonitrile, (2S,4R)-N-(benzyloxycarbonyl)-4-fluoroprolinonitrile, (2R,4S)-N-(benzyloxycarbonyl)-4-fluoroprolinonitrile, (2S,4S)-N-(tert-butoxycarbonyl)-4-fluoroprolinonitrile, (2R,4R)-N-(tert-butoxycarbonyl)-4-fluoroprolinonitrile, (2S,4R)-N-(tert-butoxycarbonyl)-4-fluoropyrrolinonitrile, (2R,4S)-N-(tert-butoxycarbonyl)-4-fluoroprolinonitrile, (2S,4S)-N-acetyl-4-fluoroprolinonitrile, (2R,4R)-N-acetyl-4-fluoroprolinonitrile, (2S,4R)-N-acetyl-4-fluoroprolinonitrile, (2R,4S)-N-acetyl-4-fluoroprolinonitrile, (2S,4S)-N-bromoacetyl-4-fluoroprolinonitrile, (2R,4R)-N-bromoacetyl-4-fluoroprolinonitrile, (2S,4R)-N-bromoacetyl-4-fluoroprolinonitrile, (2R,4S)-N-bromoacetyl-4-fluoroprolinonitrile, (2S,4S)-N-chloroacetyl-4-fluoroprolinonitrile, (2R,4R)-N-chloroacetyl-4-fluoropyrrolinonitrile, (2S,4R)-N-chloroacetyl-4-fluoroprolinonitrile, (2R,4S)-N-chloroacetyl-4-fluoroprolinonitrile, (2S,4S)-N-(9-fluorenylmethoxycarbonyl)-4-fluoroproline methyl ester, (2R,4R)-N-(9-fluorenylmethoxycarbonyl)-4-fluoroproline methyl ester, (2S,4R)-N-(9-fluorenylmethoxycarbonyl)-4-fluoroproline methyl ester, (2R,4S)-N-(9-fluorenylmethoxycarbonyl)-4-fluoroproline methyl ester, (2S,4S)-N-(benzyloxycarbonyl)-4-fluoroproline methyl ester, (2R,4R)-N-(benzyloxycarbonyl)-4-fluoroproline methyl ester, (2S,4R)-N-(benzyloxycarbonyl)-4-fluoroproline methyl ester, (2R,4S)-N-(benzyloxycarbonyl)-4-fluoroproline methyl ester, (2S,4S)-N-(tert-butoxycarbonyl)-4-fluoroproline methyl ester, (2R,4R)-N-(tert-butoxycarbonyl)-4-fluoroproline methyl ester, (2S,4R)-N-(tert-butoxycarbonyl)-4-fluoroproline methyl ester, (2R,4S)-N-(tert-butoxycarbonyl)-4-fluoroproline methyl ester, (2S,4S)-N-acetyl-4-fluoroproline methyl ester, (2R,4R)-N-acetyl-4-fluoroproline methyl ester, (2S,4R)-N-acetyl-4-fluoroproline methyl ester, (2R,4S)-N-acetyl-4-fluoroproline methyl ester, (2S,4S)-N-bromoacetyl-4-fluoroproline methyl ester, (2R,4R)-N-bromoacetyl-4-fluoroproline methyl ester, (2S,4R)-N-bromoacetyl-4-fluoroproline methyl ester, (2R,4S)-N-bromoacetyl-4-fluoroproline methyl ester, (2S,4S)-N-chloroacetyl-4-fluoroproline methyl ester, (2R,4R)-N-chloroacetyl-4-fluoroproline methyl ester, (2S,4R)-N-chloroacetyl-4-fluoroproline methyl ester, (2R,4S)-N-chloroacetyl-4-fluoroproline methyl ester, (3S)-N-(9-fluorenylmethoxycarbonyl)-3-fluoropyrrolidine, (3S)-N-(benzyloxycarbonyl)-3-fluoropyrrolidine, (3S)-N-(tert-butoxycarbonyl)-3-fluoropyrrolidine, (3S)-N-benzyl-3-fluoropyrrolidine, (3R)-N-(9-fluorenylmethoxycarbonyl)-3-fluoropyrrolidine, (3R)-N-(benzyloxycarbonyl)-3-fluoropyrrolidine, (3R)-N-(tert-butoxycarbonyl)-3-fluoropyrrolidine, and (3R)-N-benzyl-3-fluoropyrrolidine.

The starting materials, optically active hydroxypyrrolidine derivatives having formula (II) used herein are commercially available or can be prepared according to the literature or in accordance with understood principles of synthetic chemistry.

Illustrative optically active hydroxypyrrolidine derivatives, as presented by formula (II), include: methyl (2S,4R)-N-(9-fluorenylmethoxycarbonyl)-4-hydroxypyrrolidine-2-carboxylate [called as N-Fmoc-trans-4-hydroxy-L-proline methyl ester], methyl (2R,4S)-N-(9-fluorenylmethoxycarbonyl)-4-hydroxypyrrolidine-2-carboxylate [N-Fmoc-trans-4-hydroxy-D-proline methyl ester], methyl (2S,4S)-N-(9-fluorenylmethoxycarbonyl)-4-hydroxypyrrolidine-2-carboxylate [N-Fmoc-cis-4-hydroxy-L-proline methyl ester], methyl (2R,4R)-N-(9-fluorenylmethoxycarbonyl)-4-hydroxypyrrolidine-2-carboxylate [N-Fmoc-cis-4-hydroxy-D-proline methyl ester], methyl (2S,4R)-N-(benzyloxycarbonyl)-4-hydroxypyrrolidine-2-carboxylate [N-Cbz-trans-4-hydroxy-L-proline methyl ester], methyl (2R,4S)-N-(9-benzyloxycarbonyl)-4-hydroxypyrrolidine-2-carboxylate [N-Cbz-trans-4-hydroxy-D-proline methyl ester], methyl (2S,4S)-N-(benzyloxycarbonyl)-4-hydroxypyrrolidine-2-carboxylate [N-Cbz-cis-4-hydroxy-L-proline methyl ester], methyl (2R,4R)-N-(benzyloxycarbonyl)-4-hydroxypyrrolidine-2-carboxylate [N-Cbz-cis-4-hydroxy-D-proline methyl ester], methyl (2S,4R)-N-(tert-butoxycarbonyl)-4-hydroxypyrrolidine-2-carboxylate [N-Boc-trans-4-hydroxy-L-proline methyl ester], methyl (2R,4S)-N-(tert-butoxycarbonyl)-4-hydroxypyrrolidine-2-carboxylate [N-Boc-trans-4-hydroxy-D-proline methyl ester], methyl (2S,4S)-N-(tert-butoxycarbonyl)-4-hydroxypyrrolidine-2-carboxylate [N-Boc-cis-4-hydroxy-L-proline methyl ester], methyl (2R,4R)-N-(tert-butoxycarbonyl)-4-hydroxypyrrolidine-2-carboxylate [N-Cbz-cis-4-hydroxy-D-proline methyl ester], methyl (2S,4R)-N-acetyl-4-hydroxypyrrolidine-2-carboxylate [N-acetyl-trans-4-hydroxy-L-proline methyl ester], methyl (2R,4S)-N-acetyl-4-hydroxypyrrolidine-2-carboxylate [N-acetyl-trans-4-hydroxy-D-proline methyl ester], methyl (2S,4S)-N-aceyl-4-hydroxypyrrolidine-2-carboxylate [N-acetyl-cis-4-hydroxy-L-proline methyl ester], methyl (2R,4R)-N-acetyl-4-hydroxypryrrolidine-2-carboxylate [N-acetyl-cis-4-hydroxy-D-proline methyl ester], methyl (2S,4R)-N-bromoacetyl-4-hydroxypyrrolidine-2-carboxylate [N-bromoacetyl-trans-4-hydroxy-L-proline methyl ester], methyl (2R,4S)-N-bromoacetyl-4-hydroxypyrrolidine-2-carboxylate [N-bromoacetyl-trans-4-hydroxy-D-proline methyl ester], methyl (2S,4S)-N-bromoaceyl-4-hydroxypyrrolidine-2-carboxylate [N-bromoacetyl-cis-4-hydroxy-L-proline methyl ester], methyl (2R,4R)-N-bromoacetyl-4-hydroxypryrrolidine-2-carboxylate [N-bromoacetyl-cis-4-hydroxy-D-proline methyl ester], methyl (2S,4R)-N-chloroacetyl-4-hydroxypyrrolidine-2-carboxylate [N-chloroacetyl-trans-4-hydroxy-L-proline methyl ester], methyl (2R,4S)-N-chloroacetyl-4-hydroxypyrrolidine-2-carboxylate [N-chloroacetyl-trans-4-hydroxy-D-proline methyl ester], methyl (2S,4S)-N-chloroaceyl-4-hydroxypyrrolidine-2-carboxylate [N-chloroacetyl-cis-4-hydroxy-L-proline methyl ester], methyl (2R,4R)-N-chloroacetyl-4-hydroxypyrrolidine-2-carboxylate [N-chloroacetyl-cis-4-hydroxy-D-proline methyl ester], (3S)-N-(9-fluorenylmethoxycarbonyl)-3-hydroxypyrrolidine, (3S)-N-(benzyloxycarbonyl)-3-hydroxypyrrolidine, (3S)-N-(tert-butoxycarbonyl)-3-hydroxypyrrolidine, (3S)-N-benzyl-3-hydroxypyrrolidine, (3R)-N-(9-fluorenylmethoxycarbonyl)-3-hydroxypyrrolidine, (3R)-N-(benzyloxycarbonyl)-3-hydroxypyrrolidine, (3R)-N-(tert-butoxycarbonyl)-3-hydroxypyrrolidine, and (3R)-N-benzyl-3-hydroxypyrrolidine.

Arylsulfur trifluorides for use herein have thermally high stability and are easily handled (see U.S. Pat. No. 7,265,247 B1 and U.S. Pat. No. 7,381,846 B2, incorporated by reference herein for all purposes).

Illustrative arylsulfur trifluorides, as represented by formula (III), can be prepared as described in the literature [see J. Am. Chem. Soc., Vol. 82 (1962), pp. 3064-3072; Synthetic Communications, Vol. 33 (2003), pp. 2505-2509; U.S. Pat. No. 7,265,247 B1; and U.S. Pat. No. 7,381,846 B2]. Alternatively, arylsulfur trifluorides can be prepared from an arylsulfur halotetrafluoride with a reducing substance (see U.S. Patent Application No. 61/041,415). Arylsulfur trifluorides prepared from arylsulfur halotetrafluorides may be used without further purification. Each of the above references is incorporated by reference herein for all purposes.

The arylsulfur trifluorides are exemplified by, but are not limited to, phenylsulfur trifluoride, each isomer of methylphenylsulfur trifluoride, each isomer of dimethylphenylsulfur trifluoride, each isomer of trimethylphenylsulfur trifluoride, each isomer of ethylphenylsulfur trifluoride, each isomer of n-propylphenylsulfur trifluoride, each isomer of isopropylphenylsulfur trifluoride, each isomer of n-butylphenylsulfur trifluoride, each isomer of isobutylphenylsulfur trifluoride, each isomer of sec-butylphenylsulfur trifluoride, each isomer of tert-butylphenylsulfur trifluoride, each isomer of di(isopropyl)phenylsulfur trifluoride, each isomer of tri(isopropyl)phenylsulfur trifluoride, each isomer of (tert-butyl)dimethylphenylsulfur trifluoride, each isomer of (tert-butyl)(chloro)dimethylphenylsulfur trifluoride, each isomer of (tert-butyl)(dichloro)dimethylphenylsulfur trifluoride, each isomer of (methoxymethyl)phenylsulfur trifluoride, each isomer of bis(methoxymethyl)phenylsulfur trifluoride, each isomer of bis(methoxymethyl)-tert-butylphenylsulfur trifluoride, each isomer of bis(ethoxymethyl)-tert-butylphenylsulfur trifluoride, each isomer of bis(isopropoxymethyl)-tert-butylphenylsulfur trifluoride, each isomer of fluorophenylsulfur trifluoride, each isomer of chlorophenylsulfur trifluoride, each isomer of bromophenylsulfur trifluoride, each isomer of iodophenylsulfur trifluorode, each isomer of difluorophenylsulfur trifluoride, each isomer of trifluorophenylsulfur trifluoride, each isomer of tetrafluorophenylsulfur trifluoride, pentafluorophenylsulfur trifluoride, each isomer of dichlorophenylsulfur trifluoride, each isomer of dibromophenylsulfur trifluoride, each isomer of chlorofluorophenylsulfur trifluoride, each isomer of bromofluorophenylsulfur trifluoride, each isomer of chloro(methyl)phenylsulfur trifluoride, each isomer of chloro(dimethyl)phenylsulfur trifluoride, each isomer of nitrophenylsulfur trifluoride, each isomer of dinitrophenylsulfur trifluoride, each isomer of cyanophenylsulfur trifluoride, and other like compounds.

Among these arylsulfur trifluorides, phenylsulfur trifluoride, 4-methylphenylsulfur trifluoride, 2,4-dimethylphenylsulfur trifluoride, 2,5-dimethylphenylsulfur trifluoride, 2,4,6-trimethylphenylsulfur trifluoride, 4-tert-butylphenylsulfur trifluoride, 2,4,6-tri(isopropyl)phenylsulfur trifluoride, 4-tert-butyl-2,6-dimethylphenylsulfur trifluoride, 4-tert-butyl-3-chloro-2,6-dimethylphenylsulfur trifluoride, 2,6-bis(methoxymethyl)phenylsulfur trifluoride, 2,6-bis(ethoxymethyl)phenylsulfur trifluoride, 2,6-bis(methoxymethyl)-4-tert-butylphenylsulfur trifluoride, 2,6-bis(ethoxymethyl)-4-tert-butylphenylsulfur trifluoride, 4-fluorophenylsulfur trifluoride, and 4-chlorophenylsulfur trifluoride are preferable.

More preferable are phenylsulfur trifluoride, 4-methylphenylsulfur trifluoride, 4-tert-butylphenylsulfur trifluoride, 4-tert-butyl-2,6-dimethylphenylsulfur trifluoride, 2,6-bis(methoxymethyl)phenylsulfur trifluoride, 2,6-bis(methoxymethyl)-4-tert-butylphenylsulfur trifluoride, 4-fluorophenylsulfur trifluoride, and 4-chlorophenylsulfur trifluoride.

Furthermore preferable are phenylsulfur trifluoride, 4-methylphenylsulfur trifluoride, 4-tert-butyl-2,6-dimethylphenylsulfur trifluoride, 2,6-bis(methoxymethyl)phenylsulfur trifluoride, and 2,6-bis(methoxymethyl)-4-tert-butylphenylsulfur trifluoride.

The most preferable is 4-tert-butyl-2,6-dimethylphenylsulfur trifluoride because of availability, high safety, ease of handling, and high product yield.

In some embodiments, the reactions herein are carried out with addition of hydrogen fluoride (HF) or a mixture of hydrogen fluoride and an organic compound(s) since hydrogen fluoride or a mixture of hydrogen fluoride and an organic compound(s) may accelerate the fluorination reaction.

The hydrogen fluoride may be in situ generated by addition of a necessary amount of water or an alcohol such as methanol, ethanol, propanol, butanol, and so on. The water or alcohol is added into the reaction mixture, since an arylsulfur trifluoride (ArSF₃) reacts with water or an alcohol to generate hydrogen fluoride, as shown in the following reaction equations, however, this in situ generation method of hydrogen fluoride requires ArSF₃ be consumed at equimolar amounts of water or alcohol.

ArSF₃+H₂O→2HF+ArSOF

or

ArSF₃+CₙH₂ₙ₊₁OH (n=1˜4)→HF+CₙH₂ₙ₊₁F (n=1˜4)+ArSOF.

Examples of a mixture of hydrogen fluoride and an organic compound(s) include: a mixture of hydrogen fluoride and pyridine, a mixture of hydrogen fluoride and triethylamine, a mixture of hydrogen fluoride and dimethyl ether or diethyl ether, and so on. Among them, a mixture of hydrogen fluoride and pyridine and a mixture of hydrogen fluoride and triethylamine are preferable, and furthermore, a mixture of about 70 wt % hydrogen fluoride and about 30 wt % pyridine and a 3:1 molar ratio mixture of hydrogen fluoride and triethylamine are more preferable because of availability and product yield. The amount used of hydrogen fluoride or a mixture of hydrogen fluoride and an organic compound(s) is selected from a catalytic amount to a large excess.

When the starting material and/or the product are sensitive to acid conditions resulting from the formation of HF by the fluorination reaction, the reaction of this invention may be carried out in the presence of a HF-trapping agent. Preferable HF-trapping agents include metal fluorides such as lithium fluoride, sodium fluoride, potassium fluoride, cesium fluoride, and so on; carbonates such as sodium carbonate, sodium bicarbonate, potassium carbonate, potassium bicarbonate, and so on. Among them, metal fluorides are preferable.

Reactions in accordance with the invention can be carried out with one or more solvent(s). The use of solvent is preferable for mild and efficient reactions. The preferable solvents will not substantially react with the starting materials and reagents, the intermediates, and/or the final products. Suitable solvents include, but are not limited to, alkanes, halocarbons, ethers, esters, nitriles, aromatics, nitroalkanes, and so on, and mixtures thereof. Example alkanes include normal, branched, cyclic isomers of pentane, hexane, heptane, octane, nonane, decane, dodecane, undecane, and other like compounds. Illustrative halocarbons include; dichloromethane, chloroform, carbon tetrachloride, dichloroethane, trichloroethane, terachloroethane, trichlorotrifluoroethane, chlorobenzene, dichlorobenzene, trichlorobenzene, hexafluorobenzene, benzotrifluoride, and bis(trifluoromethyl)benzene; normal, branched, cyclic isomers of perfluoropentane, perfluorohexane, perfluoroheptane, perfluorooctane, perfluorononane, and perfluorodecane; perfluorodecalin; and other like compounds. Illustrative ethers include diethyl ether, dipropyl ether, di(isopropyl)ether, dibutyl ether, t-butyl methyl ether, tetrahydrofuran, dioxane, glyme (1,2-dimethoxyethane), diglyme, triglyme, and other like compounds. Illustrative esters include methyl acetate, ethyl acetate, methyl propionate, and other like compounds. Illustrative nitriles include acetonitrile, propionitrile, and other like compounds. Illustrative aromatics include benzene, toluene, xylene, and other like compounds. Illustrative nitroalkanes include nitromethane, nitroethane, and other like compounds. Among these solvents, halocarbons are the most suitable because of high product yields.

In order to obtain industrially useful yields of product in reactions in accordance with this invention, the reaction temperature can preferably be selected in the range of about −30° C. to about +100° C. More preferably, the reaction temperature can be selected in the range of about −20° C. to about +80° C. Furthermore preferably, the reaction temperature can be selected in the range of about −10° C. to about +50° C.

Reaction conditions for embodiments of this invention are optimized to obtain economically good yields of product. From about 1 mol or more, preferably about 1 mol to about 3 mol, more preferably, from about 1 mol to about 2 mol, furthermore preferably, from about 1 mol to about 1.6 mol of arylsulfur trifluoride (formula III) are combined with 1 mol of an optically active hydroxypyrrolidine derivative (formula II) to obtain a good yield of optically active fluoropyrrolidine derivative (formula I) (several Examples are provided herein).

Note that the reaction time for embodiments of this invention varies dependent upon reaction temperature, and the types and amounts of substrates, reagents, and solvents. As such, reaction time is generally determined as the amount of time required to complete a particular reaction, but can be from about 0.1 h to about a few weeks, preferably, within a week.

The following examples will illustrate the present invention in more details, but it should be understood that the present invention is not deemed to be limited thereto.

### EXAMPLES

The following examples are provides for illustrative purposes only and are not intended to limit the scope of the invention. Table 1 provides formula numbers, names, and structures for reference when reviewing the following examples.

TABLE 1
Optically Active Fluoropyrrolidine Derivatives (Formulas Ia-If):

| Formula | | |
|---|---|---|
| Number | Name | Structure |
| Ia | (2S,4S)-N-Fmoc-4- fluoroprolinonitrile | 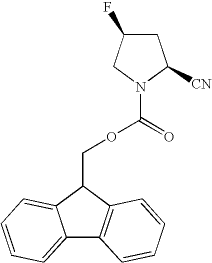 |
| Ib | (2S,4S)-N-Cbz-4- fluoroprolinonitrile | 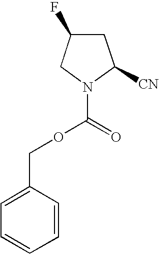 |
| Ic | (2S,4S)-N-Boc-4- fluoroprolinonitrile | 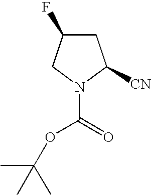 |
| Id | (2S,4S)-N-Fmoc-4- fluoroproline methyl ester | 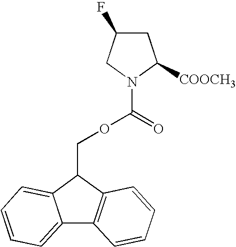 |
| Ie | (2S,4S)-N-Boc-4- fluoroproline methyl ester | 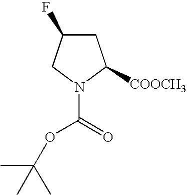 |
| If | (2S,4R)-N-Boc-4- fluoroproline methyl ester | 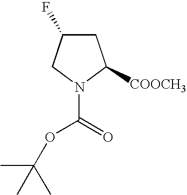 |

Fmoc = 9-fluorenylmethoxycarbonyl.
Cbz = benzyloxycarbonyl.
Boc = tert-butoxycarbonyl.

### Example 1

### Preparation of (2S,4S)-N-Fmoc-4-fluoroprolinonitrile (Ia)

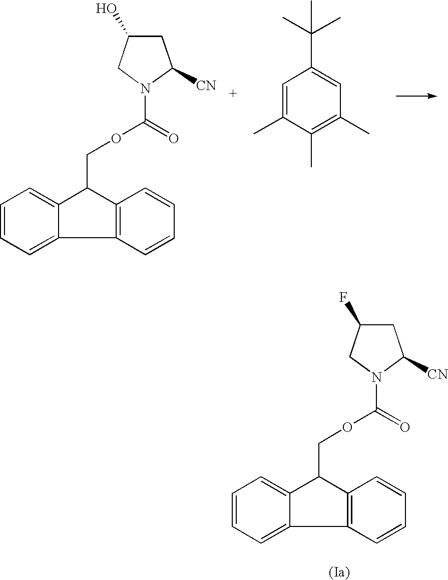

(2S,4R)-N-Fmoc-4-hydroxyprolinonitrile (1.67 g, 5.01 mmol) and 3 mL of dry dichloromethane were placed in a fluoro polymer (PFA) vessel. The mixture was cooled to near 0° C. on an ice bath. Into the mixture was slowly added a solution of 4-tert-butyl-2,6-dimethylphenylsulfur trifluoride (1.88 g, 7.51 mmol) in 2 ml of dry dichloromethane. After complete addition, the reaction mixture was stirred for 1 hour. Then the ice bath was removed and the mixture was stirred at room temperature for 60 hours. ¹⁹F NMR analysis of the reaction mixture showed that (2S,4S)-N-Fmoc-4-fluoroprolinonitrile (Ia) was produced in an almost quantitative yield (99%). The reaction mixture was evaporated to dryness and the resulting residue was dissolved in 4 mL of a 1:1 mixture of dichloromethane and ether. Into the solution, an excess amount of anhydrous pentane was added, giving a solid. The solid was again dissolved in 4 mL of a 1:1 mixture of dichloromethane and ether, and an excess amount of anhydrous pentane was added. The resulting solid was then thin-layer chromatographed to give 1.43 g (85%) of the pure product (Ia). The spectral data of (Ia) are shown by the following: ¹⁹F-NMR (CDCl₃) δ −174.63 (m): ¹H-NMR (CDCl₃) δ 2.15-2.85 (m, 2H), 3.4-4.0 (m, 2H), 4.15-4.85 (m, 4H), 5.30 (d, 1H, J=51.6 Hz, 4-H), 7.27-7.85 (m, 8H): ¹³C-NMR (CDCl₃) 8 (as a mixture of two rotamers) a major rotamer; 36.97 (d, J=21.0 Hz), 45.85, 52.86 (d, J=24.6 Hz), 68.18, 91.98 (d, J=179.9 Hz), 117.97, 120.20, 125.22, 128.02, 141.45, 143, 153.97; a minor rotamer; 38.03 (d, J=21.7 Hz), 45.38, 47.15, 53.20 (d, J=24.6 Hz), 68.58, 90.93 (d, J=179.2 Hz), 118.16, 120.20, 125.05, 127.33, 141.45, 143, 153.54.

### Examples 2-9

### Preparation of Optically Active Fluoropyrrolidine Derivatives (Ib)˜(If)

Optically active fluoropyrrolidine derivatives (Ib)˜(If) were prepared by reaction of optically active hydroxypyrrolidine derivatives with an arylsulfur trifluoride in a similar manner as described in Example 1. The optically active hydroxypyrrolidine derivatives and the arylsulfur trifluorides employed in Examples 2-9 are shown in Table 2. The results and reaction conditions are also shown in Table 2 together with those of Example 1. In Examples 3, 6, and 9, the reactions were conducted in the presence of sodium fluoride as a HF-trapping agent (which is shown as an additive in Table 2).

TABLE 2
Preparation of Optically Active Fluoropyrrolidine Derivatives (Ia)~(If)

| | | | | Conditions | | |
|---|---|---|---|---|---|---|
| | | | | and | | |
| Ex | (II) | (III) | Solvent | Additive | Product | Yield* |
| 1 | 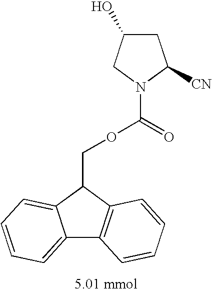 | 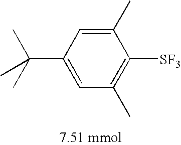 | CH₂Cl₂ 5 mL | ~0° C., 1 h, and then r.t., 60 h | 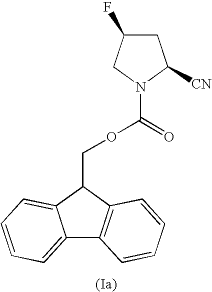 | 99% (85%) |
| 2 | 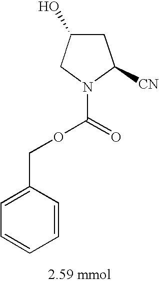 | 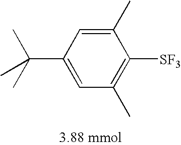 | CH₂Cl₂ 5 mL | ~0° C., 1 h, and then r.t., 72 h | 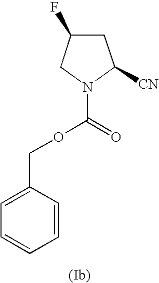 | 99% (79%) |
| 3 | 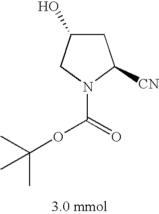 | 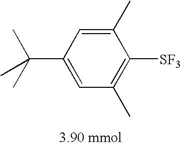 | CH₂Cl₂ 8 mL | ~0° C., 1 h, and then r.t., 60 h Additive; NaF (4.5 mmol) | 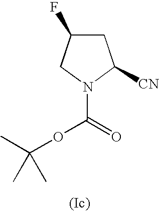 | 80% (64%) |
| 4 | 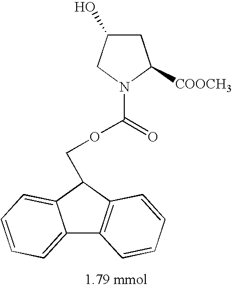 | 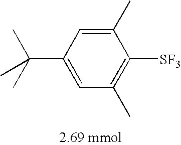 | CH₂Cl₂ 5 mL | ~0° C., 1 h, and then r.t., 60 h | 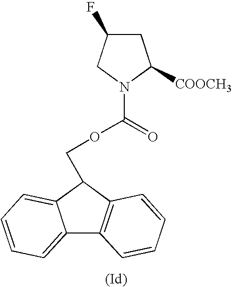 | 69% |
| 5 | 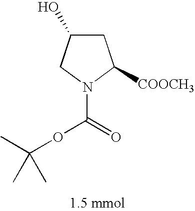 | 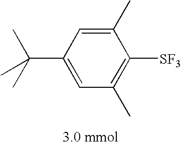 | CH₂Cl₂ 6 mL | r.t. 21 h | 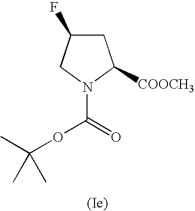 | 57% |
| 6 | 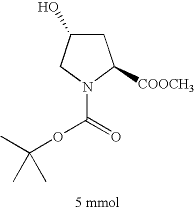 | 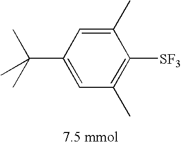 | CH₂Cl₂ 5 mL | ~0° C.→r.t., 0.5 h and then r.t., 65 h Additive; NaF (5 mmol) | 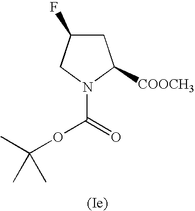 | 74% |
| 7 | 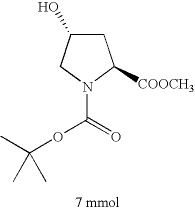 | 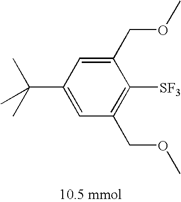 | CH₂Cl₂ 5 mL | ~0° C.→r.t., 0.5 h and then r.t., 65 h | 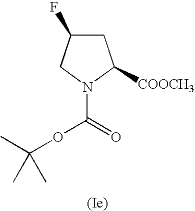 | 65% |
| 8 | 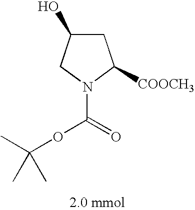 | 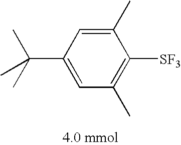 | CH₂Cl₂ 6 mL | r.t., 21 h | 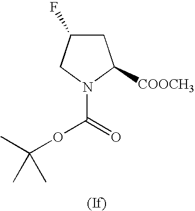 | 69% |
| 9 | 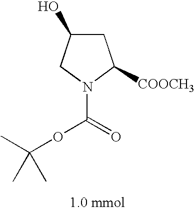 | 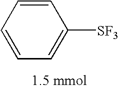 | CH₂Cl₂ 4 mL | r.t., 15 h Additive; NaF (6 mmol) | 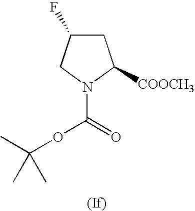 | 64% |

*Yields are determined based on ¹⁹F NMR analysis, and yields given in parentheses are isolated yields.

Spectral data of (2S,4S)-N-Fmoc-4-fluoroprolinonitrile (Ia) are shown in Example 1, and those of products (Ib)-(Id) are shown below. (2S,4S)-N-Boc-4-fluoroproline methyl ester (Ie) and (2S,4R)-N-Boc-4-fluoroproline methyl ester (If) were identified by spectral analysis and comparison with authentic samples.

(2S,4S)-N-Cbz-4-fluoroprolinonitrile (Ib): ¹⁹F-NMR (CDCl₃) δ −174.93 (m); ¹H-NMR (CDCl₃) δ 2.1-2.7 (m, 2H), 3.4-4.0 (m, 2H), 4.69 (t, J=10.3 Hz, 1H), 5.05-5.4 (m, 3H), 7.2-7.5 (m, 5H): ¹³C-NMR (CDCl₃) 8 (as a mixture of two rotamers) a major rotamer; 36.91 (d, J=21.7 Hz), 45.87, 52.93 (d, J=23.8 Hz), 67.95, 92.19 (d, J=178.5 Hz), 118.28, 128.28, 128.5, 128.73, 135.93, 154.06; a minor rotamer; 37.82 (d, J=20.9 Hz), 45.40, 53.25 (d, J=24.6 Hz), 68.10, 91.16 (d, J=178.5 Hz), 118.40, 128.19, 128.5, 128.73, 135.93, 153.44.

(2S,4S)-N-Boc-4-fluoroprolinonitrile (Ic): ¹⁹F-NMR (CDCl₃) δ −175.09 (m): ¹H-NMR (CDCl₃) 8 (as a mixture of two rotamers) 1.42 (s, ˜⅓x9H), 1.46 (s, ˜⅔x9H), 2.15-2.65 (m, 2H), 3.4-3.9 (m, 2H), 4.55-4.7 (m, 1H), 5.26 (d, J=51.6 Hz, 1H): ¹³C-NMR (CDCl₃) 8 (as a mixture of two rotamers) a major rotamer; 28.30, 31.03, 37.73 (d, J=20.9 Hz), 45.54, 52.62 (d, J=23.8 Hz), 82.07, 91.08, (d, J=178.5 Hz), 118.39, 152.72; a minor rotamer; 28.30, 31.03, 36.98 (d, J=21.7 Hz), 45.33, 52.98 (d, J=24.7 Hz), 81.70, 92.17 (d, J=179.2 Hz), 118.39, 153.26.

(2S,4S)-N-Fmoc-4-fluoroproline methyl ester (Id); ¹⁹F-NMR (CDCl₃) δ −172.63 (m): ¹H-NMR (CDCl₃) 8 (as an about 54:46 mixture of two rotamers) 2.2-2.7 (m, 2H), 3.6-4.0 (m, 5H, including two CH₃ singlet peaks at 3.67 as a minor rotamer and 3.76 as a major rotamer), 4.15-4.7 (m, 4H), 5.20 (dm, J=52.3 Hz, 0.54H), 5.25 (dm, J=52.6 Hz, 0.46H), 7.2-7.9 (m, 8H): ¹³C-NMR (CDCl₃) 8 (as a mixture of two rotamers) a major rotamer; 37.79 (d, J=21.7 Hz), 47.25, 52.67, 53.33 (d, J=24.6 Hz), 57.84, 67.75, 92.20 (d, J=177.7 Hz), 120.11, 127.20, 127.85, 141.38, 143.82, 144.13, 155.64, 171.80; a minor rotamer; 36.70 (d, J=21.7 Hz), 47.32, 52.67, 53.59 (d, J=24.6 Hz), 57.54, 67.63, 91.20 (d, J=177.7 Hz), 120.11, 127.20, 127.85, 141.38, 143.82, 144.13, 154.40, 171.80.

As shown above, according to the present invention, substantially complete optically active fluoropyrrolidine derivatives are obtained in high yield from optical active hydroxypyrrolidine derivatives by stereospecific fluorination with inversion at the 3- or 4-position of pyrrolidine ring. The arylsulfur trifluoride used in this invention are highly thermal stable and are easily prepared and handled. Therefore, the method of the present invention provides a useful and unexpectedly improved method for a large scale industrial production of optically active fluoropyrrolidine derivatives. These materials are useful fluorinated intermediates for therapeutics and other like materials.

All references including publications and patents are incorporated by reference herein for all purposes.

While the invention has been particularly shown and described with reference to some of embodiments, it would be understood by those skilled in the art that changes in the form and details may be made to the embodiments disclosed herein without departing from the spirit and scope of the invention and that the embodiments disclosed herein are not intended to act as limitations on the scope of the claims.

What is claimed is:

1. A method for preparing an optically active fluoropyrrolidine derivative having a formula (I) as follows: 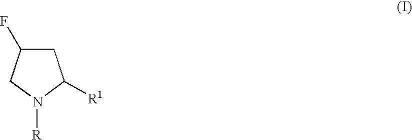
the method comprising reacting an optically active hydroxypyrrolidine derivative having a formula (II) with an arylsulfur trifluoride having a formula (III); 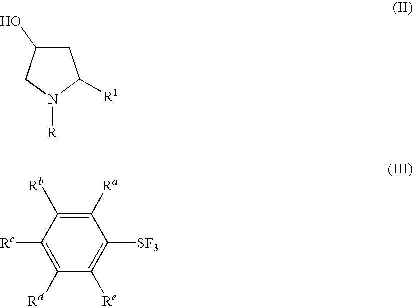
in which R is a protecting group for an amino group, R¹ is a hydrogen atom, a cyano group, or a COOR² group, in which R² is a protecting group for a carboxyl group; and R^{a}, R^{b}, R^{c}, R^{d}, and R^{e} each is independently a hydrogen atom, a halogen atom, a substituted or unsubstituted alkyl group having one to 10 carbon atoms, a nitro group, or a cyano group.

2. The method of claim 1 wherein R¹ is a cyano group or a COOR² group.

3. The method of claim 1 wherein the substituted or unsubstituted alkyl group having one to 10 carbon atoms has one to 4 carbon atoms.

4. The method of claim 1 further comprising reacting hydroxypyrrolidine derivative having a formula (II) with an arylsulfur trifluoride having a formula (III) in the presence of hydrogen fluoride or a mixture of hydrogen fluoride and an organic compound(s).

5. The method of claim 1 further comprising reacting hydroxypyrrolidine derivative having a formula (II) with an arylsulfur trifluoride having a formula (III) in the presence of a HF-trapping agent.

6. The method of claim 1 wherein the arylsulfur trifluoride is selected from a group consisting of phenylsulfur trifluoride, 4-methylphenylsulfur trifluoride, 2,4-dimethylphenylsulfur trifluoride, 2,5-dimethylphenylsulfur trifluoride, 2,4,6-trimethylphenylsulfur trifluoride, 4-tert-butylphenylsulfur trifluoride, 2,4,6-tri(isopropyl)phenylsulfur trifluoride, 4-tert-butyl-2,6-dimethylphenylsulfur trifluoride, 4-tert-butyl-3-chloro-2,6-dimethylphenylsulfur trifluoride, 2,6-bis(methoxymethyl)phenylsulfur trifluoride, 2,6-bis(ethoxymethyl)phenylsulfur trifluoride, 2,6-bis(methoxymethyl)-4-tert-butylphenylsulfur trifluoride, 2,6-bis(ethoxymethyl)-4-tert-butylphenylsulfur trifluoride, 4-fluorophenylsulfur trifluoride, and 4-chlorophenylsulfur trifluoride.

7. The method of claim 1 wherein the arylsulfur trifluoride is selected from a group consisting of phenylsulfur trifluoride, 4-methylphenylsulfur trifluoride, 4-tert-butylphenylsulfur trifluoride, 4-tert-butyl-2,6-dimethylphenylsulfur trifluoride, 2,6-bis(methoxymethyl)phenylsulfur trifluoride, 2,6-bis(methoxymethyl)-4-tert-butylphenylsulfur trifluoride, 4-fluorophenylsulfur trifluoride, and 4-chlorophenylsulfur trifluoride.

8. The method of claim 1 wherein the arylsulfur trifluoride is selected from a group consisting of phenylsulfur trifluoride, 4-methylphenylsulfur trifluoride, 4-tert-butyl-2,6-dimethylphenylsulfur trifluoride, 2,6-bis(methoxymethyl)phenylsulfur trifluoride, and 2,6-bis(methoxymethyl)-4-tert-butylphenylsulfur trifluoride.

9. The method of claim 1 wherein the arylsulfur trifluoride is 4-tert-butyl-2,6-dimethylphenylsulfur trifluoride.

10. The method of claim 1 wherein the reacting an optically active hydroxypyrrolidine derivative with an arylsulfur trifluoride is performed between −30° C. and +100° C.

11. The method of claim 1 wherein the reacting an optically active hydroxypyrrolidine derivative with an arylsulfur trifluoride is performed between −20° C. and +80° C.

12. The method of claim 1 wherein the molar ratio range of optically active hydroxypyrrolidine to arylsulfur trifluoride is between 1:1 and 1:3.

13. The method of claim 1 wherein the molar ratio range of optically active hydroxypyrrolidine and arylsulfur trifluoride is between 1:1 and 1:2.

14. The method of claim 1 further comprising reacting an optically active hydroxypyrrolidine derivative with an arylsulfur trifluoride in a solvent selected from a group consisting of alkanes, halocarbons, ethers, esters, nitriles, aromatics, and nitroalkanes.

15. The method of claim 1 further comprising reacting an optically active hydroxypyrrolidine derivative with an arylsulfur trifluoride in a solvent, wherein the solvent is a halocarbon.
